(19) United States
(12) Patent Application Publication
(10) Pub. No.: US 2013/0267822 A1
(43) Pub. Date: Oct. 10, 2013

(54) Probe

(71) Applicant: Yusuke Mimura, Hino-shi (JP)

(72) Inventor: Yusuke Mimura, Hino-shi (JP)

(21) Appl. No.: 13/992,618

(22) PCT Filed: Nov. 22, 2011
(86) PCT No.: PCT/JP2011/076868

§ 371 Date: Jun. 7, 2013

(30) Foreign Application Priority Data
Dec. 7, 2010 (JP) 2010-272443

(51) Int. Cl. A61B 1/00

(52) U.S. Cl. CPC A61B 1/00163
USPC 600/407

(57) ABSTRACT

Provided is a probe and a measurement system which uses the probe. According to one implementation, the probe includes a radiated-light measurement optical system and an oblique-viewing imaging device. The radiated-light measurement optical system irradiates illumination light onto a measurement target region on a biological tissue. The oblique-viewing imaging device has an angular field α and a forward tilt angle β defined by a center axis of a field of view and a radial direction of the probe. The radiated-light measurement optical system has an optical element that determines a direction of emission of the illumination light, and a forward tilt angle γ is defined by the direction of emission and the radial direction of the probe. The oblique-viewing imaging device and the optical element are positioned such that the measurement target region lies within the angular field.

### TECHNICAL FIELD

The present invention relates to a probe including an optical system that irradiates illumination light onto a measurement target region of a biological tissue, receives the light radiated from the measurement target region, and measures the radiated light, and to a probe further including an imaging device.

### BACKGROUND ART

Special diagnosis probes have been developed that irradiate illumination light, such as excitation light, onto a measurement target region of a biological tissue and detect the radiated light, such as fluorescent light, that is generated from the illumination light at the biological tissue or a drug injected in advance into a biological subject. The special diagnosis probes have been used in diagnosis of degeneration or diseases, such as cancer, in the biological tissue (for example, the type of the disease and the extent of invasion).

Such a probe includes an optical system, such as an optical fiber or a prism, for guiding the illumination light from a light source device, irradiating the illumination light onto a measurement target region on a biological subject, receiving the light radiated from a pathological region, and guiding the light to an analyzing device.

Preferably, the probe is inserted into the body while the direction of the insertion (direction of direct view) is being visually confirmed. The measurement target region by the probe is often positioned close to a side of the probe; thus, it is desirable to acquire an image of a side including the measurement target region (direction of side view) that can be used for diagnosis.

A special diagnosis probe will now be discussed that detects radiation light, such as fluorescent light, and is equipped with an imaging device that images an image in the direction of insertion (direction of direct view) and the side (direction of side view).

Patent Literature 1 describes a side-viewing fluorescence endoscope that performs fluoroscopy at a predetermined distance in multiple directions by rotating the inner circumferential surface of a luminal subject.

Patent Literature 2 describes an oblique-viewing endoscope that has a view ranging from the direction of insertion (direction of direct view) to the side (direction of side view) as a result of tilting the viewing direction of the imaging device.

In Patent Literature 2, a super-wide angle lens is placed at the tip of the endoscope to maintain a view angle of approximately 180 degrees with the important view direction as the center. According to this document, an optical image that has passed through the super-wide lens is received by a solid-state imaging element in an imaging camera, is converted to electrical signals in the element, and is sent to an image processing device. The image processing device calculates the viewing direction in a predetermined range from the central direction instructed by an operator with a joy stick, calculates the positions of pixels on the solid-state imaging element corresponding to the calculated viewing direction, reads image signals corresponding to the calculated pixels from an image memory, and outputs the image signals to a monitor device. In this way, distortion in an image due to the super-wide lens is corrected. Thus, multiple directions can be observed with a single endoscope that does not have a mechanically movable part.

### PATENT ART DOCUMENT

### Patent Literature

Patent Literature 1: Japanese Patent Application Laid-Open Publication No. 2008-142346
Patent Literature 2: Japanese Patent Application Laid-Open Publication No. H10-290777

### DISCLOSURE OF THE INVENTION

### Problems to be Solved by the Invention

As described in Patent Literature 1, the examination of fluorescent light with a side-viewing endoscope involves an inconvenient insertion process because the direction of insertion (direction of direct view) cannot be visually confirmed.

A special diagnosis probe that includes a measurement system, for example, for fluorescent light and that accommodates an imaging optical system having an oblique field of view, such as that described in Patent Literature 2, entails the following issues.

The measurement system, for example, for fluorescent light and the imaging optical system that guides an optical image transmitted through a super-wide angle lens to a solid-state imagining element cannot be positioned inside a small-diameter probe. The super-wide angle lens may preclude a decrease in the diameter of a tip portion of the probe, and thus, the tip portion of the probe may have an increased diameter to displace the imaging element and the super-wide angle lens inside the tip portion of the probe.

This is undesirable because of the need of image processing for distortion correction in an image caused by the super-wide angle lens and image processing for outputting, to a monitor, image signals that correspond to a predetermined range instructed by the operator. Complicated operation may also be required.

An object of the present invention, which has been conceived in light of the issues concerning the background art, is to provide a probe that includes an optical system for irradiating illumination light onto a measurement target region on a biological tissue and receiving light radiated from the measurement target region and that measures the radiated light, the probe having a tip portion accommodating an oblique-viewing imaging device while preventing the diameter from increasing, the probe acquiring a high-quality image with a range of view including the direction of insertion (direction of direct view) and the measurement target region by the oblique-viewing imaging device, without special image processing, and the probe satisfactorily measuring radiated light.

### Means for Solving the Problem

The present invention according to claim 1 provides a solution for solving the issues described above, the probe including:

a radiated-light measurement optical system that irradiates illumination light onto a measurement target region on a biological tissue and receives light radiated from the measurement target region; and

an oblique-viewing imaging device that has an angular field α and a forward tilt angle β defined by a center axis of a field of view and a radial direction of the probe,

wherein

the oblique-viewing imaging device comprises a wide-angle lens and an imaging element,

the radiated-light measurement optical system has an optical element that determines a direction of emission of the illumination light and a direction of photoreception of the radiated light rearward to the oblique-viewing imaging device, and a forward tilt angle γ is defined by the direction of emission and the radial direction of the probe,

the oblique-viewing imaging device and the optical element are accommodated in a tip portion of the probe and are rotatable around a longitudinal direction axis of the tip portion,

the angular field α is in a range of 90°<α<110°,

the forward tilt angle β is in a range of 20°<β<35°,

the forward tilt angle γ is in a range of 5°≦γ≦15°, and

the oblique-viewing imaging device and the optical element are positioned such that the measurement target region lies within the angular field.

The present invention according to claim 2 provides the probe of claim 1, wherein a distance L in the radial direction between a center of the angular field of the oblique-viewing imaging device and the measurement target region satisfies L>(D−S−tan γ)/[tan(α/2−β)+tan γ] (1), where D is a distance in an axial direction between the center of the angular field of the oblique-viewing imaging device and the emission point of the illumination light from the optical element, and S is a distance in the radial direction.

### Advantageous Effect of the Invention

According to the present invention, a forward tilt angle β within the range mentioned above allows for a probe including a tip portion accommodating an oblique-viewing imaging device while preventing the diameter from increasing; an angular field a within the range mentioned above allows for the acquisition of a high-quality image without special image processing; and α, β, γ, and Expression 1 within the ranges mentioned above allow for the acquisition of a high-quality image of a range of view including the direction of insertion (direction of direct view) and the measurement target region by the oblique-viewing imaging device; and the forward tilt angle γ allows for satisfactory measurement of radiated light by preventing light reflecting from the glass cover on the outer circumference of the probe and highly efficiently receiving the radiated light.

### BRIEF DESCRIPTION OF THE DRAWINGS

FIG. 1 is a schematic side view of a probe according to an embodiment of the present invention.

FIG. 2 is the same schematic side view as FIG. 1 including various parameters.

FIG. 3 is a graph representing the relationship between the angle of emitted light from a perpendicular line to the surface of a specimen and the efficiency of the intensity of the returning light to the intensity of the emitted light.

FIG. 4 is a graph representing the relationship between the forward tilt angle β of an oblique-viewing imaging device and the radial dimension x of the oblique-viewing imaging device.

FIG. 5 is a graph representing the relationship between the forward tilt angle β of the oblique-viewing imaging device and the forward view distance fa of the oblique-viewing imaging device.

FIG. 6 is a graph representing the relationship between the forward tilt angle β of the oblique-viewing imaging device and the rear view distance fb of the oblique-viewing imaging device.

### EMBODIMENT FOR CARRYING OUT THE INVENTION

An embodiment of the present invention will now be described with reference to the drawings. The embodiment according to the present invention described below should not limit the invention. In the following embodiment, a probe for fluorescence photometry is exemplified.

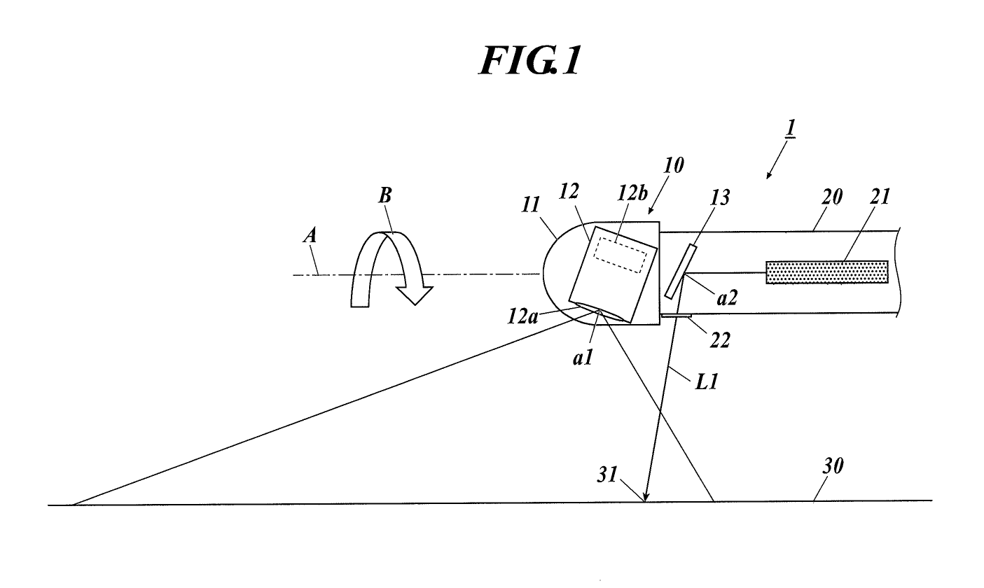

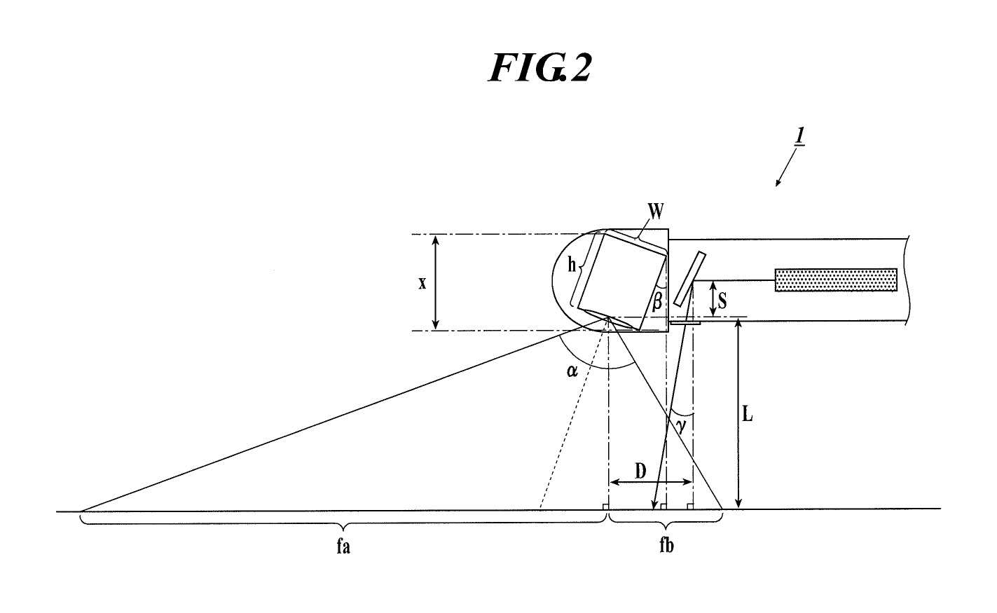

FIGS. 1 and 2 illustrate an area including a tip portion 10 of a probe 1 according to this embodiment.

A tip jacket portion 11 is disposed at the end of the tip portion 10 of the probe 1. The tip jacket portion 11 is composed of a transparent material formed to have a dome-shaped tip. An oblique-viewing imaging device 12 is accommodated in the tip jacket portion 11. The oblique-viewing imaging device 12 is an integrated unit of a wide-angle lens 12a and an imaging element 12b which includes a CCD.

A mirror 13 is disposed rearward of the oblique-viewing imaging device 12. The oblique-viewing imaging device 12 and the mirror 13 can turn in the direction indicated by arrow B around the longitudinal axis A. That is, the oblique-viewing imaging device 12 and the mirror 13 are integrated by using an appropriate frame member and are connected to a rotation actuator. The rotation actuator is disposed in the tip portion 10 or at the base of the probe 1. In the latter case, a freely bendable torque tube that transmits torque is used as a power transmission member.

The rear end of the tip jacket portion 11 is connected to a freely bendable outer tube 20, which constitutes the outer circumferential surface of the probe. An optical fiber pair 21 extends through the outer tube 20. The optical fiber pair 21 includes an illuminative optical fiber and a photoreceptive optical fiber. The illuminative optical fiber and the photoreceptive optical fiber extend from the base of the probe 1 to the vicinity of the tip portion 10 such that their tips are disposed toward the mirror 13. The video-signal output cable and the power supply cable of the oblique-viewing imaging device 12 pass through the outer tube 20.

A glass cover 22 is disposed on the side of the probe 1 as a window for fluorescence photometry. Excitation light L1 emitted from the illuminative optical fiber of the optical fiber pair 21 is reflected by the mirror 13, is emitted to the outside through the glass cover 22, and is irradiated onto a measurement target region 31 on a biological tissue 30. The radiated light from the measurement target region 31, which contains the light reflected at the measurement target region 31 and the fluorescent light generated at the measurement target region 31, is taken into the probe 1 through the glass cover 22, is reflected at the mirror 13, and enters the core of the photoreceptive optical fiber of the optical fiber pair 21. A focusing lens may be disposed between the mirror 13 and the fibers including the illuminative optical fiber and the photoreceptive optical fiber for efficient light guiding.

The mirror 13 is an optical element that determines the direction of emission of the excitation light L1 (illumination light) and the direction of reception of the radiated light in the radiated-light measurement optical system. The mirror 13 may be replaced by a prism.

The center of the angular field of the oblique-viewing imaging device 12 is represented by a1, and the emission point of the excitation light L1 (illumination light) on the mirror 13 is represented by a2.

A balloon, which is mounted on the outer circumference of the probe 1 and can freely expand or contract, holds the tip portion 10 at the center of the lumen during measurement. The probe is held at the center of the lumen by the balloon and measures the inner surface of the lumen by rotating while emitting illumination light.

The base of the probe 1, which is disposed outside the subject when the probe 1 is inserted inside the subject, is connected to a base unit of the measurement system including the probe 1.

The base unit includes an excitation-light source, a spectroscope that receives the light guided through the photoreceptive optical fiber, and an arithmetic unit that carries out analysis and video processing.

In a broad sense, fluorescent light is excess energy emitted as electromagnetic waves when electrons in a subject are excited by energy absorbed by the subject irradiated with X-rays, ultraviolet rays, or visible rays and return to the ground state. Fluorescent light having a wavelength different from that of the excitation light (reference light) that generates the fluorescent light is returned. Wavelength spectrometry is performed on the radiated light that enters a spectroscope, and the resulting spectral distribution is analyzed by the arithmetic unit to measure the intensity of generated fluorescent light. The arithmetic unit performs data processing, such as recording the fluoroscopy data in a data recorder and displaying the data on an image display. Images imaged by the oblique-viewing imaging device 12 are displayed on the image display. The arithmetic unit processes data to combine an image overlapping the fluoroscopy data and the surface image of the biological tissue 30, which includes the measurement target region 31 and is imaged by the oblique-viewing imaging device 12, records the combined image in the data recorder, and displays the combined image on the image display.

As illustrated in FIGS. 1 and 2, the angular field of the oblique-viewing imaging device 12 is represented by α, the forward tilt angle between the oblique-viewing imaging device 12 and the radial direction of the probe 1 is represented by β, and the forward tilt angle between the direction of emission of the excitation light L1 and the radial direction of the probe 1 is represented by γ.

As described in detail below, the oblique-viewing imaging device 12 and the mirror 13 are positioned such that the measurement target region 31 lies within the angular field α, where the angular field α is in the range of 90°<α<110°, the forward tilt angle β is in the range of 20°<β<35°, and the forward tilt angle γ is in the range of 5°≦γ≦5°.

As illustrated in FIGS. 1 and 2, the forward view distance from the center of the angular field a1 is represented by fa; the rear view distance from the center of the angular field a1 is represented by fb; the distance in the direction of the axis A between the center of the angular view a1 and the emission point a2 is represented by D; the radial distance is represented by S; and the radial distance between the center of the angular view a1 and the measurement target region 31 is represented by L. The thickness of the oblique-viewing imaging device 12 in the radial direction at β=0 is represented by h, and the width of the oblique-viewing imaging device 12 in the direction of the axis A at β=0 is represented by W. The actual radial dimension of the oblique-viewing imaging device 12 is represented by x.

The present invention enables simultaneous observation of a measurement target region of a luminal wall and in the direction of insertion, without dedicated image processing by the oblique-viewing imaging device 12. An exemplary configuration will now be described in steps.

### (1-1) Angular Field α of Oblique-Viewing Imaging Device 12

Referring to FIGS. 1 and 2, the angular field α needs to be a certain degree for simultaneous observation in the directions of insertion and measurement. An increase in the angular field α causes an inevitable increase in the size of the wide-angle lens 12a, thus, causing an increase in the size of the tip jacket portion 11 accommodating the oblique-viewing imaging device 12. The angular field α should be determined in consideration of the size of the oblique-viewing imaging device 12.

### (1-2) Size of Oblique-Viewing Imaging Device 12 and Forward Tilt Angle β

As described in Section (1-1) above, an increase in the angular field α causes an inevitable increase in the size of the oblique-viewing imaging device 12. The degree of tilt to position the oblique-viewing imaging device 12 in an oblique-viewing state is also a major issue; if the oblique-viewing imaging device 12 tilts as illustrated in FIGS. 1 and 2, the tip portion 10 will be larger than that of a normal direct viewing or side-viewing imaging device. The tilt angle of the oblique-viewing imaging device 12, i.e., the forward tilt angle β, must be determined in consideration of the size of the oblique-viewing imaging device 12 such that a sufficient field of view is acquired in the direction of the insertion.

The size of the tip portion 10 must be determined in consideration of the insertion procedure of the probe, i.e., nasal insertion, oral insertion, or anal insertion for colonoscopy. In general, the diameter (φ) for nasal insertion is 5.5 or less, oral insertion is 11 or less, and anal insertion is 14 or less.

### (1-3) Forward Tilt Angle γ in Direction of Emission

It is effective to emit emitting light (L1) at a forward tilt angle in a certain degree instead of emitting the light perpendicular to the luminal surface (30), as illustrated in FIGS. 1 and 2. This is for the following two reasons. One is that the measurement target region 31 lies within the field of view of the oblique-viewing imaging device 12 if the emitting light (L1) is emitted at a forward angle toward the oblique-viewing imaging device 12. The other is that if emitting light (L1) is emitted in a perpendicular state, the light reflected on the glass cover 22 is detected as the returning light, and by emitting the emitting light (L1) in a tilted state, the light reflected on the glass cover 22 is hardly detected.

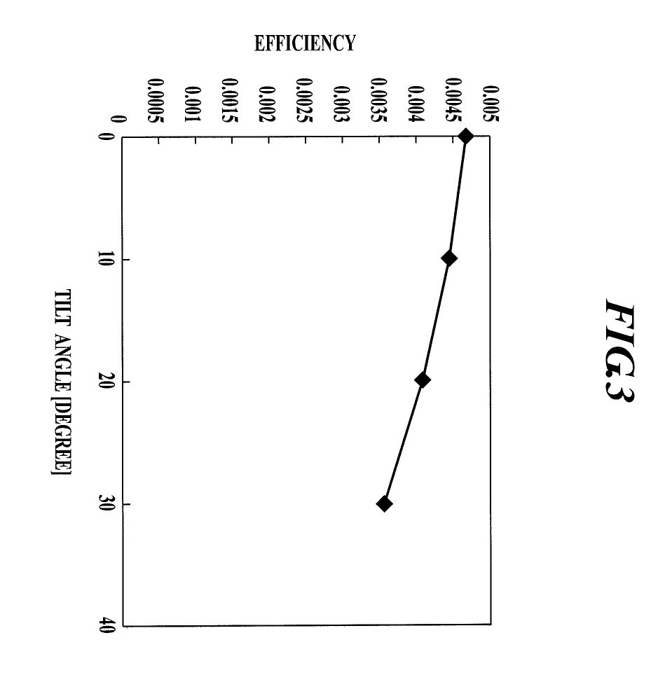

A large forward tilt angle γ reduces the intensity of the returning light. Thus, the forward tilt angle γ must be determined in consideration of these reasons. FIG. 3 represents a simulation result determining the relationship between the angle of the emitting light from a perpendicular line to the surface of the specimen and the efficiency of the returning light to the intensity of the emitted light.

### (1-4) Distance to Target Luminal Wall

The measurement target of the probe 1 is a luminal wall of a biological subject. If the distance to the luminal wall is far, the measurement target region 31 can be easily captured by the oblique-viewing imaging device 12, whereas if the distance is near, it is difficult to capture the measurement target region 31. The visibility of the emitted light must be checked in advance for the distance to the target using Expression 1 described above. Example organs having a measurement target luminal wall includes esophagus, small intestine, and large intestine. The diameter of the esophagus and the small intestine is 20 mm, and the diameter of the large intestine is in the range of 40 to 60 mm.

### (1-5) Positional Relationship Between Oblique-Viewing Imaging Device 12 and Light-Emitting Unit

The positional relationship between the oblique-viewing imaging device 12 and the light-emitting unit (mirror 13) should be considered in order to capture the measurement target region 31 in the field of view of the oblique-viewing imaging device 12. Although the two components are preferably positioned close to each other, the positional relationship thereof should be determined in consideration of the size of the oblique-viewing imaging device 12.

A detailed example configuration in consideration of the components described in Sections (1-1) to (1-5) will now be described. The example configuration described below is a probe that is nasally inserted to measure the esophagus.

### (2-1) Angular Field α of Oblique-Viewing Imaging Device 12

The angular field α must be at least 90° for observation in the direction of insertion and observation of the measurement target region 31. From this view point, a large angular field α is preferred. A large angular field α, however, inevitably requires a large wide-angle lens 12a or multiple wide-angle lenses 12a, resulting in a large oblique-viewing imaging device 12. Another issue in a large angular field α is low image quality. In general, there are nasal endoscopes having an angular field of 140° for the camera. Unlike such typical endoscopes, the present invention requires the installation of a measurement optical system including a light-emitting unit. Thus, compared with typical nasal endoscopes, the oblique-viewing imaging device 12 has to be installed in a small mounting space and obliquely to capture the direction of the insertion and the measurement target region 31 in the field of view. Consequently, the oblique-viewing imaging device 12 must be smaller than typical nasal endoscopes. Thus, the angular field α is preferably in the range of 90° to 110°, where 90° is the minimum necessary view angle and 110° is an angle that maintains the image quality of the camera to a certain extent while keeping the size of the oblique-viewing imaging device 12 small. Accordingly, the angular field α is 100° in this example configuration.

### (2-2) Forward Tilt Angle β of Oblique-Viewing Imaging Device 12

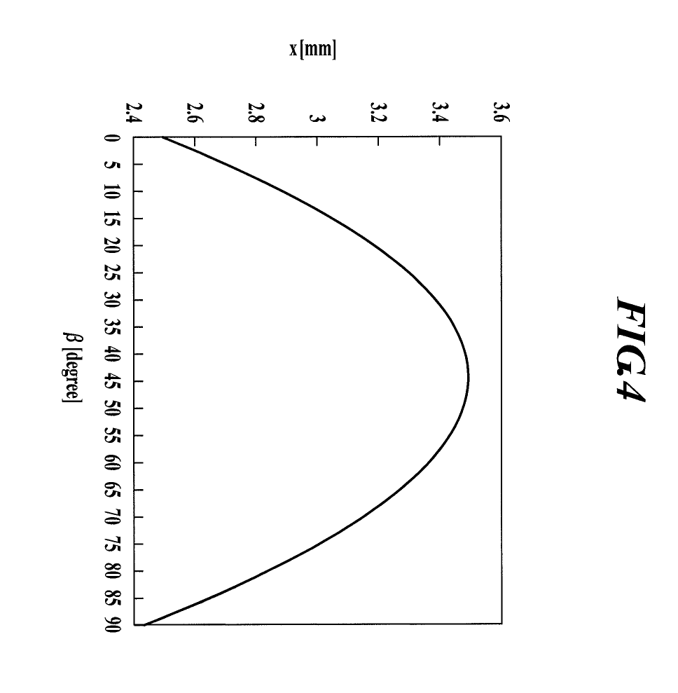

The dimensions of the oblique-viewing imaging device 12 of this example configuration are: h=2.49 mm and W=2.45 mm. FIG. 4 is a graph representing the relationship between the forward tilt angle β and the radial dimension x of the oblique-viewing imaging device 12. FIG. 4 shows that the dimension x is maximized near 45°. Thus, the forward tilt angle β should not be near 45° and should be determined in consideration of the forward view distance fa, the observation of the measurement target region 31, and the positional relationship of the emitting light (L1). The relationship between the forward tilt angle β and the dimension x is represented by Expression 2:

x=h·cos β+w·sin β (2)

The forward view distance fa and the rearward view distance fb for observing the measurement target region 31 can be derived from the forward tilt angle β and the angular field α and represented by Expressions 3 and 4:

fa=L·tan(α/2+β) (3)

fb=L·tan (α/2−β) (4)

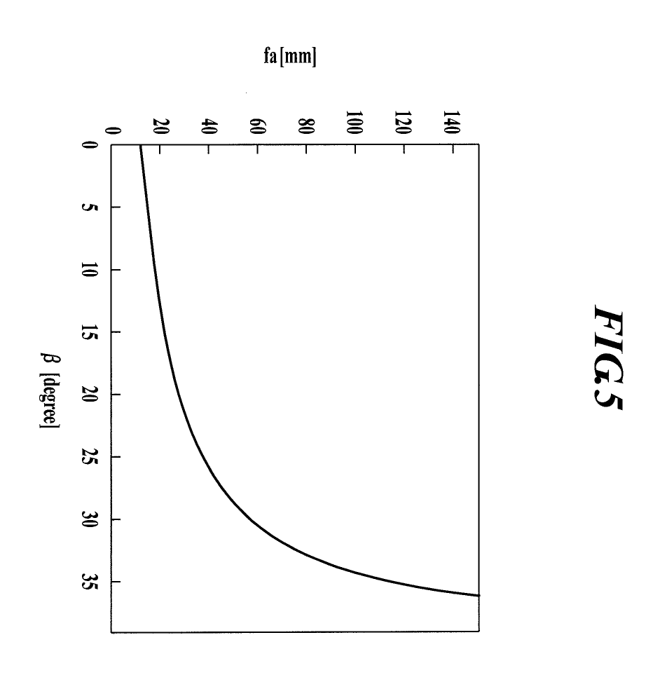

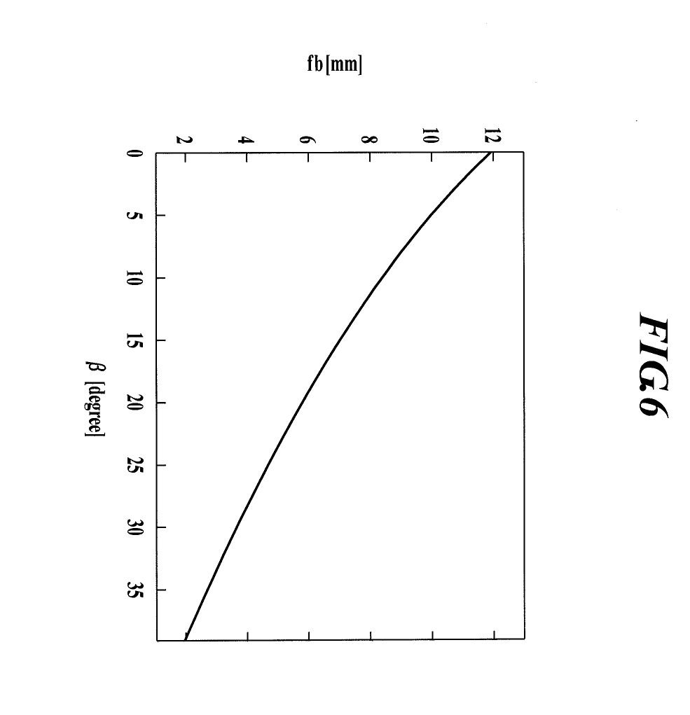

In this example configuration, L=10 mm because the esophagus is the target of measurement, and the angular field α is 100° as mentioned above. These values are assigned in Expressions 3 and 4, which are plotted as graphs. The graph representing the variation in the forward view distance fa to the forward tilt angle β is shown in FIG. 5, and the graph representing the variation in the rearward view distance fb to the forward tilt angle β is shown in FIG. 6. In this example configuration, the target forward view distance fa is 50 mm because the direction of insertion of the probe can be confirmed and the target organ is not saclike, such as the stomach, and thus, the forward tilt angle β is 29°. As a result, fa=51.4 mm and fb=3.83 mm.

### (2-3) Relative Positional Relationship Between Oblique-Viewing Imaging Device 12 and Light-Emitting Unit

The relative positional relationship between the oblique-viewing imaging device 12 (center of angular view a1) and the light-emitting unit (emission point a2 of mirror 13) is defined by the distance D and the distance S. The positional relationship is extremely important for capturing the measurement target region 31 of the emitting light (L1) in the field of view of the oblique-viewing imaging device 12. The condition for the measurement target region 31 to lie within the angular field α is represented by Expression 1:

L>(D−S·tan γ)/[tan(α/2−β)+tan γ] (1)

A distance D as small as possible and a distance S as large as possible are preferred to satisfy the Expression 1. The distance cannot be decreased to a predetermined value or less because the oblique-viewing imaging device 12 is tilted and the mirror 13 is disposed next to the oblique-viewing imaging device 12. The distance S cannot be increased to a predetermined value or more because its upper limit is determined by the thickness of the probe. In this example configuration, D=1.4 mm and S=1.3 mm.

In this example configuration, α=100°, β=29°, and γ=10°, as mentioned above. These values satisfy Expression 1.

As described above, the forward tilt angle β and the forward tilt angle γ are determined from the angular field α.

### (3) Summary

The effective ranges of major parameters a, 13, and γ are summarized as follows.

### [Angular Field α]

If the angular field α is 90° or less, the direction of insertion and the measurement target region 31 do not simultaneously lie within the field of view of the camera. An angular field α of 110° or more increases the space dedicated to the wide-angle lens 12a, which causes an increase in the size of the oblique-viewing imaging device 12, and lowers the camera performance, such as image quality; thus, the preferred angular field α is in the range of 90° to 110°.

### [Forward Tilt Angle β]

The values predominantly affected by the forward tilt angle β are the forward view distance fa and rearward view distance fb of the oblique-viewing imaging device 12 and the radial dimension x of the oblique-viewing imaging device 12.

Referring to FIG. 4, the difference between the maximum and minimum values of the dimension x is approximately 1 mm. This difference is large for nasal insertion in consideration of the predetermined diameter (φ) of 5.5 or less. Referring to FIG. 5, it is possible to see that the forward view distance fa of the camera increases near 30°. A forward view distance fa of 30 mm or more is required to capture the direction of the insertion of the probe within the imaging range to facilitate the insertion of the probe and to observe luminal organs; thus, the preferred forward tilt angle β is in the range of 20° to 35°.

### [Forward Tilt Angle γ]

The preferred angle γ of the emitting light is in the range of 5° to 15°, as mentioned above.

In the embodiment described above, excitation light is irradiated onto the measurement target region, and fluorescent light generated from the excitation light is received. Alternatively, scattered light or Raman scattered light generated from the illumination light may be received. In such a case, the degeneration or diseases, such as cancer, in the biological tissue can be diagnosed.

### INDUSTRIAL APPLICABILITY

The probe according to the present invention can be used to examine the condition of a biological tissue, such as the pathological condition, through optical measurement of the biological tissue.

### DESCRIPTION OF REFERENCE NUMERALS

1 probe
10 tip portion
11 tip jacket portion
12 oblique-viewing imaging device
12a wide-angle lens
12b imaging element
13 mirror
20 outer tube
21 optical fiber pair
22 glass cover
30 biological tissue
31 measurement target region
A longitudinal axis
a1 center of angular view
a2 emission point
L1 emitting light
α angular field
β forward tilt angle
γ forward tilt angle

1. A probe comprising:
a radiated-light measurement optical system that irradiates illumination light onto a measurement target region on a biological tissue and receives light radiated from the measurement target region; and
an oblique-viewing imaging device that has an angular field α and a forward tilt angle β defined by a center axis of a field of view and a radial direction of the probe,
wherein
the oblique-viewing imaging device comprises a wide-angle lens and an imaging element,
the radiated-light measurement optical system has an optical element that determines a direction of emission of the illumination light and a direction of photoreception of the radiated light rearward to the oblique-viewing imaging device, and a forward tilt angle γ is defined by the direction of emission and the radial direction of the probe,
the oblique-viewing imaging device and the optical element are accommodated in a tip portion of the probe and are rotatable around a longitudinal direction axis of the tip portion,
the angular field α is in a range of 90°<α<110°,
the forward tilt angle β is in a range of 20°<β<35°,
the forward tilt angle γ is in a range of 5°≦γ≦15°, and
the oblique-viewing imaging device and the optical element are positioned such that the measurement target region lies within the angular field.

2. The probe of claim 1, wherein a distance L in the radial direction between a center of the angular field of the oblique-viewing imaging device and the measurement target region satisfies L>(D·S tan γ)/[tan(α/2−β)+tan γ] (1), where D is a distance in an axial direction between the center of the angular field of the oblique-viewing imaging device and the emission point of the illumination light from the optical element, and S is a distance in the radial direction.

3. The probe of claim 1, wherein a tip jacket portion composed of a transparent material formed to have a dome-shaped tip is disposed at an end of the tip portion.

4. The probe of claim 3, wherein a tube is connected to a rear end of the tip jacket portion.

5. The probe of claim 4, wherein an illuminative optical fiber and a photoreceptive optical fiber are provided in the tube.

6. The probe of claim 4, wherein a power supply cable of the oblique-viewing imaging device is provided in the tube.

7. The probe of claim 4, wherein a video-signal output cable of the oblique-viewing imaging device is provided in the tube.

8. The probe of claim 1, wherein the oblique-viewing imaging device is integrated with the optical element.

9. The probe of claim 1, further comprising a rotation actuator connected to the oblique-viewing imaging device and the optical element.

10. The probe of claim 1, wherein a window for measurement by the radiated-light measurement optical system is disposed on a side of the probe.

11. The probe of claim 1, wherein the optical element is a mirror.

12. The probe of claim 1, wherein the optical element is a prism.

13. The probe of claim 1, wherein a balloon which can freely expand or contract is mounted on an outer circumference of the probe.

14. The probe of claim 1, wherein a dimension of the oblique-viewing imaging device in a radial direction of the probe is a diameter of 5.5 mm or less.

15. A measurement system comprising:
a probe of claim 1; and
a base unit where a base of the probe is connected to,
wherein the base unit includes a light source of the illumination light, a spectroscope that receives input of light received by the radiated-light measurement optical system, and an arithmetic unit.

16. The measurement system of claim 15, wherein the arithmetic unit carries out data processing to combine an image overlapping an image imaged by the oblique-viewing imaging device with measured data.

17. The measurement system of claim 15, wherein the light received by the radiated-light measurement optical system is a fluorescent light caused by the illumination light.

18. The measurement system of claim 15, wherein the light received by the radiated-light measurement optical system is a scattered light caused by the illumination light.

19. The measurement system of claim 15, wherein the light received by the radiated-light measurement optical system is a Raman scattered light caused by the illumination light.
